(19) United States
(12) Patent Application Publication
(10) Pub. No.: US 2004/0161452 A1
(43) Pub. Date: Aug. 19, 2004

(54) Drug-dispensing dressing and composition for treating onychomycosis

(76) Inventor: MIchael G. Petit, Montecito, CA (US)

Correspondence Address: Michael G. Petit, P.O. Box 91929, Santa Barbara, CA, 93190-1929, US

(21) Appl. No.: 10/370,129

(22) Filed: Feb. 18, 2003

(51) Int. Cl. A61K 9/70

(52) U.S. Cl. 424/445

(57) ABSTRACT

A drug-dispensing dressing for administering a fluid containing an antifungal agent to a fungus infected nail on a toe or a finger. In a preferred embodiment, the dressing includes: (a) a reservoir comprising a substantially flat, fluid-absorbant pad having a first hole therein; and (b) an elongate strip of a fluid impermeable material having a central portion with a second hole therein. The elongate strip has a skin-facing surface coated with a layer of adhesive. The skin-facing surface of the central portion of the strip is affixed to the fluid-absorbant pad with the second hole overlying the first hole. The dressing is affixed to a toe and the reservoir, which is rechargeable, is charged with a fluid antifungal agent introduced dropwise or injected through the hole where the fluid is absorbed and disbursed by the pad and slowly released to the underlying nail. The dressing may further include a removable, preferably elastic, barrier layer disposed over the holes. In another embodiment, the reservoir is attached to the inner surface of an elastic tube to underlie a hole in the tube. The tubular dressing is stretched and placed over the affected toe with the reservoir overlying the infected nail and the antifungal agent introduced into the reservoir via the hole.

### BACKGROUND OF THE INVENTION

1. Field of the Invention

The present invention relates to a composition, method and drug-dispensing dressing for the treatment of fungal infections of the nails, particularly toenails.

2. Prior Art

Onychomycosis, also called ringworm of the nails, or tinea unguium, is a fungus infection of the nails causing thickening, roughness and splitting usually caused by Trichophyton rubrum, Trichophyton tonsurans, Epidermophyton floccosum or Trichophyton mentagrophytes. Once these microorganisms establish themselves within or under a nail, eradication with current over-the-counter (OTC) antifungal agents is difficult and recurrences of the disease can be costly and time consuming. Antifungal agents that are formulated for topical application, such as undecylenic acid and tolnaftate, are effective for treating fungal infections of the skin but are ineffective for treating fungal infections of the nail.

Current treatments include prolonged and sustained application of topical fungicidal creams and/or solutions in combination with systemic fungal treatment drugs such as griseoflilvin, terbinafine, and itraconazole. Some of the systemic treatments have undesirable side effects such as nausea, headache, photosensitivity, gastrointestinal intolerance, elevated liver enzymes, and undesirable drug interactions, making the process of eradication difficult and troublesome.

Stettendorf et al., in U.S. Pat. No. 4,721,724, disclose an antifungal composition containing clotrimazole, urea, a lipophilic wetting agent and a consistency imparting component that may be applied to an infected nail, under an occlusive dressing, to remove the infected nail atraumatically.

Garafano, in U.S. Pat. No. 6,495,153, discloses an antifungal treatment for use on skin comprising, in combination, an antifungal component and a skin-treating component. The skin-treating component either absorbs moisture on the skin to make an undesirable environment for the fungus, increases the permeability of the skin to allow the antifungal component to penetrate, promotes healing of the skin or creates an unpleasant, oily environment for the fungus. The antifungal component is a combination of goldenseal root powder with ipe roxo powder and pokeroot powder. The skin-treating component is either a skin softening mixture, such as D-alpha tocopherol and olive oil, or is a dehydrator, such as arrowroot and ball clay. A further combined antifungal and aromatic component preferably comprises lavender oil, and may also comprise teatree oil. The teaching is limited to the treatment of fungal infections of the skin and is formulated as either a powder or a cream.

Reeves, in U.S. Pat. No. 6,391,879, discloses a therapeutic solution and application procedure for curing and preventing nail fungus infections comprising a combination of an anti-fungal agent with DMSO in a anhydrous solution of Polyglycol. The DMSO acts as a solvent so that active anti-fungal agents such as Tolnaftate can be delivered directly to fungal infected areas under and around the nail. The DMSO and Tolnaftate are dissolved in a viscous solution of polyglycol so that the combined ingredients may be applied directly to the affected areas with superior penetration. The therapeutic solution is formulated by dissolving the Tolnaftate into the DMSO, and then combining the DMSO and polyglycol solutions to form the therapeutic solution. The solution is repeatedly applied to infected nails over a 4-6 week period.

Sun, et al., in U.S. Pat. No. 6,042,845, the contents of which patent being incorporated herein by reference thereto, discloses a method for the treatment of fungal diseases in nails, which comprises the topical administration to the nail and, if desired, also to the surrounding skin, of (1) a sulfhydryl containing amino acid or a derivative thereof, the pharmaceutically acceptable salts or esters thereof, or stereoisomers thereof, (2) urea, (1) and (2) being administered in an amount sufficient to enhance the permeation of antifungal drugs through nail tissue, either prior to or, preferably, concurrently with the topical administration to the nail of (3) an effective amount of an antifungal drug. There is also disclosed a bandage adapted for the topical administration of medication to the nail. The bandage comprises a T-shaped adhesive backing, and a flexible pad having an impervious backing and a nail-shaped cavity backed by the impervious backing. The nail-shaped cavity contains absorptive means having absorbed therein urea and a sulfhydryl containing amino acid or a derivative thereof, a pharmaceutically acceptable salt or ester thereof, or a stereoisomer thereof.

Tea Tree oil has been reported to posess antifungal activity. Tea Tree oil is isolated by distilling the oil contained in the stems and leaves of the paperbark tree Melaleuca alternafolia. Tea tree oil is volatile and, when applied to a nail as a neat oil, quickly evaporates. Lee, in U.S. Pat. No. 6,413,555, discloses an antifungal composition for treating onychmycoses that includes the essential oils of tea tree, lavender and eucalyptus. The composition comprises between about 10 and about 17 ml. of tea tree; between about 10 and about 17 ml. of lavender oil, and between about 1 and about 3.5 ml. of eucalyptus oil in a total of 30 ml. of liquid. The liquid mixture is applied to the affected area once or twice a day for a period of at least two months until the infection is cured.

The forgoing treatments for fungus infections of the nails, and, in the teaching of the '153 patent, for treatment of fungal infections of the skin, have not enjoyed success in the marketplace for the treatment of onychomycosis. One possible reason is that compliance with the requirement for consistent sustained application of antifungal agent to the infected nail is difficult to achieve. Another reason is that the methods of delivering the currently available compositions are, in many cases, ineffective, even with patient compliance. There remains a need for an effective method, device and composition for treating onychomycosis that is safe, easy to self-administer and rapid acting thereby reducing the treatment time and improving patient compliance and does not require the removal of the infected nail.

### SUMMARY

It is an object of the present invention to provide a composition and a method for using the composition for the treatment of onychomycoses.

It is a further object of the invention to provide a device for the sustained delivery of an antimycotic agent into the site of a fungal infection within a nail.

The above objectives of the invention are met by the provision of a fluid-impermeable, refillable elastic dressing, preferably having the shape and elasticity of a large finger cot, having a reservoir on a nail-facing surface thereof and affixed thereto. The reservoir contains a fluid antifungal composition comprising, for example, either emu oil, teat tree oil, lavender oil, or an antimycotic agent such as undecylenic acid, tolnaftate or clotrimazole in either emu oil, tea tree oil, lavender oil or a mixture thereof. The dressing is applied to the affected digit either by friction or by adhesive means such that the reservoir overlies the infected nail. The reservoir is an absorbant pad, preferably moleskin or an open-celled foam pad, that is saturated with the therapeutic (antifungal) fluid composition. The absorbant pad may be any sheet material capable of absorbing and releasing the therapeutic composition. The tight fit of the dressing to the digit, which, in one embodiment, is further secured and reinforced by the placement of an elastic barrier such as a finger cot or similar device over the dressing. The elastic barrier holds the reservoir in contact with the infected nail to permit continuous transfer of the antimycotic agent to the nail and prevent the migration of the antimycotic agent away from the nail such as, for example, by wicking or evaporation. The reservoir may be refilled in situ by the introduction of the fluid antifungal composition through an aperture in the dressing until the reservoir is saturated.

The features of the invention believed to be novel are set forth with particularity in the appended claims. However the invention itself, both as to organization and method of operation, together with further objects and advantages thereof may be best understood by reference to the following description taken in conjunction with the accompanying drawings.

### BRIEF DESCRIPTION OF THE DRAWINGS

FIG. 1 is a longitudinal cross-sectional view of a nail attached to a toe showing the anatomical relationship between the nail and the supporting structure.

FIG. 2 is a perspective view of a drug delivery device in accordance with a first preferred embodiment of the invention operable for delivering an antimycotic agent to an infected nail.

FIG. 3 is a longitudinal cross-sectional view of the drug delivery device of FIG. 2.

FIG. 4 is a transverse cross-sectional view of the drug delivery device of FIGS. 2 and 3 positioned on a toe to deliver antimycotic agent to an infected nail.

FIG. 5 is a longitudinal side view of a second preferred embodiment of a drug-dispensing dressing having a refillable reservoir affixed thereto adapted to be adhesively affixed to a toe such that the reservoir overlies the infected nail.

FIG. 6 is a top view of the drug-dispensing dressing of FIG. 5.

FIG. 7 is a bottom view of the drug-dispensing dressing of FIG. 5.

FIG. 8 is an end view of a dressing in accordance with the embodiment of FIGS. 5-7 affixed to a toe with the reservoir overlying the infected nail and an elastic barrier envelope applied over the dressing.

FIG. 9 is a longitudinal side view of a drug-dispensing dressing in accordance with a third preferred embodiment of the drug delivery device of the present invention.

FIG. 10 is a top view of the dressing illustrated in FIG. 9.

FIG. 11 is a bottom view of the skin-facing surface of the embodiment of the dressing illustrated in FIGS. 9 and 10.

FIG. 12 is a perspective view of a fluid-impermeable elastic envelope that may be applied either over any of the dressings illustrated in FIGS. 4-9, or over a coating of emu oil applied directly to the infected nail.

FIG. 13 is a perspective view of a drug-dispensing dressing in accordance with FIGS. 9-11 prior to being applied to a toe having a nail thereon that is infected with a fungus.

FIG. 14 is a perspective view of a drug-dispensing dressing in accordance with FIGS. 9-11 applied to a toe with the fill hole overlying the fungus infected nail, the Figure showing an antifungal agent being introduced into the reservoir through an access hole in the dressing, and a fluid impermeable barrier readied for positioning on the toe to overlie the dressing after the antifungal agent is added to the reservoir.

FIG. 15 is a longitudinal cross-sectional view of a drug-dispensing dressing in accordance with FIGS. 2 and 3 further comprising an elastically deformable flap affixed to the dressing disposed to cover the hole. The flap is opened to expose the hole for filling the reservoir with an antifungal agent.

FIG. 16 is a top perspective view of a drug-dispensing dressing in accordance with FIGS. 5-7 or 9-11 further comprising an elastically deformable flap affixed to the dressing disposed to cover the hole. The flap is opened to expose the hole for filling the reservoir with an antifungal agent. After filling the reservoir, the flap returns to its normally closed position to overlie the hole.

### DESCRIPTION OF THE PREFERRED EMBODIMENTS

The terms “antimycotic agent” and “antifungal agent” are used interchangeably herein to mean any composition that either kills or inhibits the growth of a pathogenic fungus infecting the nail of a digit. The term “pathogenic fungi”, as used herein, includes, but is not limited to, the fungal organisms associated with onychmycosis such as T. rubrum and T. mentagrophytes. The term “fungicidal agent”, as used herein, means a therapeutic agent that kills pathogenic fungi and their spores.

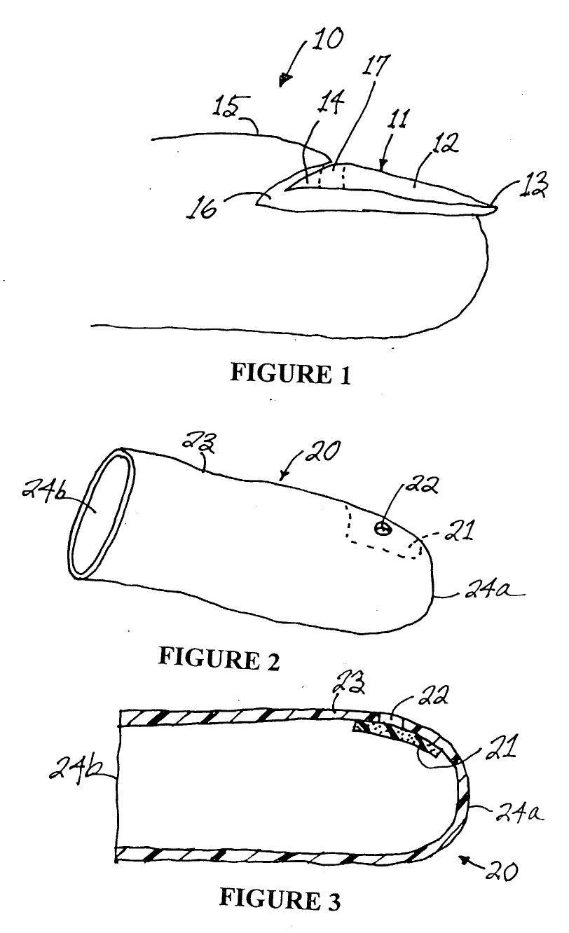

With reference now to FIG. 1, a toe 10 comprises a nail or unguis 11 having a nail body 12, a free edge 13 and a nail root 14. The nail body 12 is a horny, keratinized material referred to as the stratum lucidum. The root 14 fastens the nail 11 to a finger or toe by fitting into a groove in the skin 15. A nail matrix (germinal layer) 16 lies beneath and posterior to the nail body 12 and the nail root 14. The nail matrix 16 is vascularized and firmly connects the nail to the underlying connective tissue. The nail 11 grows in length by proliferation of germ cells in the nail root 14 and grows thicker by proliferation of cells in the portion of the nail matrix beneath the lunula 17. The site of fungal infection in the nail may be limited to the horny, keratinized nail body 12 or it may further involve the nail matrix 16. In most cases of onychmycosis, the fungal mycelium penetrate and infect the tough stratum lucidum and it is difficult to deliver a therapeutically effective dosage of an antifungal agent to the site of infection.

As pointed out by Sun, et al. (i.b.i.d) the nail body is too thick and too dense for drugs to penetrate at a practical rate. Although the composition of the nail is similar to the stratum corneum of the skin in that it is derived from epidermis, it is mainly composed of hard keratin (highly disulfide-linked) and is approximately 100-fold thicker than stratum corneum. In order to deliver a sufficient amount of an antifungal agent into the nail body, the permeability of the nail body to the antifungal agent needs to be enhanced, or, more preferably, an antifungal agent that is able to readily penetrate the nail body may be used. Prior art compositions comprising antifungal agents have attempted to increase the permeability of the nail by incorporating hydrating agents such as urea in the composition. Since there is little aqueous fluid in the nail body relative to the stratum corneum, an antifungal agent comprised of an oil, either alone or in combination with a hydrophobic antifungal agent, may provide a therapeutic composition effective for treating onychomycosis.

Several biogenic oils have received attention in recent years, mostly anecdotal, for their potentially therapeutic properties. Most notably, emu oil, lavender oil and tea tree oil have all been reported, though anecdotally, as possessing antimycotic properties. Emu oil is comprised principally of triglycerides and is a nearly neutral lipid. Emu oil is extracted from a fatty pad on the back of the emu, an Australian bird, and is commercially available (Uniquely Emu Products, Inc., Ottawa, Ill. 61530), at a relatively low cost, as a pale yellow oil having a long shelf life relative to other animal oils. It is currently used as a skin permeation enhancer for the transdermal delivery of nonsteroidal anti-inflammatory agents. Further, the long shelf life of emu oil suggests that it may exhibit certain antimicrobial activity. If the antimicrobial activity includes antifungal activity, the relatively high viscosity of pure emu oil (relative to other biogenic oils), at least at room temperature, renders it difficult to permeate an absorbant pad such as is conventionally used in dressings with the emu oil prior to applying it to the nail. Tea tree oil, which has a high terpene content and is volatile, is miscible in emu oil. The mixture has a relatively low viscosity (compared with pure emu oil), is less volatile and is readily absorbed and released from a pad-like reservoir.

While many antifungal medicaments suitable for both topical and systemic administration are known, the use of topically applied antifungal agents for the treatment of onychmycoses has not been successful. Prior art attempts to develop antifungal compositions for the treatment of onychomycoses have been directed toward increasing the permeability of the nail to the antifungal agents. It is equally important, however, to administer the composition to the nail in such a manner that the composition is not preferentially absorbed by competitive absorbants such as socks before it can fully penetrate the nail. U.S. Pat. No. 6,042,845 provides a bandage that is designed to maintain contact between an infected nail and a reservoir containing a therapeutic agent in combination with a permeation enhancer comprising sulfhydryl and urea. The reservoir is precharged prior to use and is not refillable.

A drug-dispensing dressing comprising a refillable reservoir in accordance with a first preferred embodiment of the present invention is indicated at numeral 20 in FIGS. 2 and 3. The dressing 20 comprises a hollow, fluid-impermeable elastic body 23 having an absorbant reservoir 21 affixed to, and integral with, a portion of an inner surface of the body 23 adjacent the closed distal end 24a of the body 23. A hole 22 in the body 23 overlies the reservoir 21. A proximal end 24b of the dressing 20 is open to allow the dressing to be pulled over a toe or finger having an infected nail thereon.

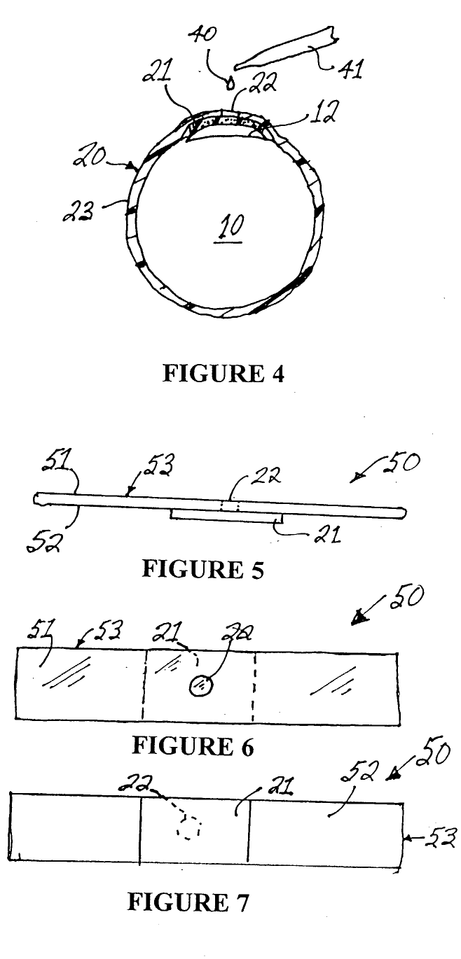

In operation, the dressing is stretched and pulled over the toe of finger until the reservoir 21 is positioned over the nail as shown in FIG. 4. The elastic body 23 is preferably constructed from either silicone, polyurethane or latex elastomer, and may be formed either by repeatedly dip-coating a digit-shaped mandrel with uncured elastomer and curing between coats, or by molding. The reservoir 21 may be affixed to the body 23 either during molding of the device or adhered to the body by adhesive means or by vulcanization after the body is formed. Once the dressing 20 is correctly positioned on the affected digit 10 with the reservoir 21 in contact with the infected nail 12, an antifungal fluid agent 40 is introduced into the reservoir 21 through the hole 22 and is taken up by the reservoir until the pad is saturated therewith. The elastic body portion 23, being impermeable to the medicament 40, minimizes or prevents competitive absorbtion of the antifungal agent 40 from the reservoir by clothing (not shown) or the like, and maximizes transfer of the antifungal agent 40 to the nail 12. Since permeation of the nail by the antifungal agent progresses slowly, additional antifungal agent 40 may be periodically introduced into the reservoir to maintain saturation thereof. In a variation of the first preferred embodiment 20, the hole 22 in the elastic body portion 23 may further extend downwardly through the reservoir 21.

A second preferred embodiment 50 of a drug-dispensing dressing in accordance with the present invention is illustrated in side view in FIG. 5, top view in FIG. 6 and bottom view in FIG. 7. The dressing 50 comprises a strip 53 of a fluid-impermeable material, preferably a vinyl polymer or copolymer, having a top surface 51 and a bottom surface 52 with a hole 22 disposed in the center thereof. The bottom surface 52 of the strip 53 preferably has a layer of adhesive coated thereon that is operable for adhering the strip 53 to the skin. A pad comprising a material that readily absorbs the antifungal agent of choice such as, for example, moleskin, is affixed to the strip 53 to underlie the hole 22 and forms the reservoir 21. The hole 22 in the strip 53 does not extend through the pad comprising the reservoir 21.

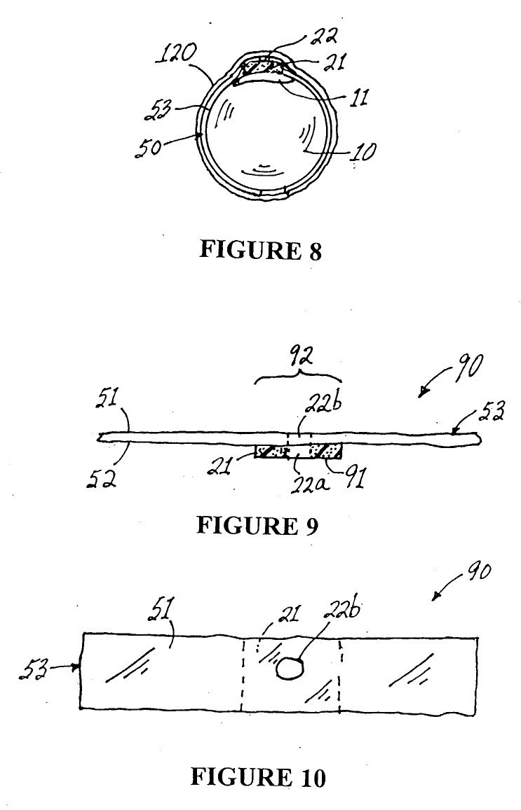

In operation, the strip 53 is affixed to the digit 10 such that the reservoir 21 overlies the nail as illustrated in end view in FIG. 8. Antifungal agent 40 is introduced into the reservoir dropwise through the hole 22 until the reservoir is saturated. An elastic barrier 120 (FIG. 12) is stretched and pulled over the end of the digit to overlie the dressing 50 and relaxed. The elastic barrier 120 serves two purposes: first, it compresses the reservoir 21 against the nail 11 to assure intimate contact therebetween. Second, the barrier 120 prevents fluid antifungal agent from exiting the reservoir via the hole 22 to be competitively absorbed by, for example, a sock.

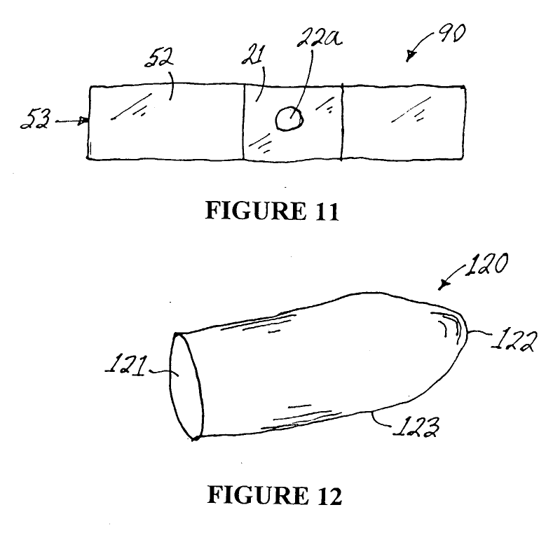

In the drug-dispensing dressings 20 and 50, a portion of the reservoir 21 underlies the hole 22 and antifungal agent may be transferred from the reservoir to absorbant clothing overlying the hole 22 if an elastic barrier 120 is not interposed therebetween to prevent wicking. A third and most preferred embodiment of a drug-dispensing dressing in accordance with the invention that reduces or eliminates the need for an elastic barrier 120 is shown at 90 in FIGS. 9-11. The drug-dispensing dressing 90 comprises a strip 53 of a fluid-impermeable material, again preferably a vinyl polymer or copolymer, having a top surface 51 and a bottom surface 52 with a hole 22 disposed in the center thereof. The bottom surface 52 of the strip 53 preferably has a layer of adhesive coated thereon that is operable for adhering the strip 53 to the skin. A pad comprising a material that readily absorbs the antifungal agent of choice such as, for example, moleskin, is affixed to the strip 53 to underlie the hole 22 and forms the reservoir 21. However, unlike the embodiment indicated at numeral 50, the third preferred embodiment 90 includes a hole in the reservoir 21 that underlies, and is coextensive with, the hole 22 in the strip 53. When antifungal agent is introduced dropwise through the hole 22, the antifungal agent migrates laterally to saturate the pad and forms a film on the surface of the infected nail. The antifungal agent is slowly absorbed by the nail. Since the strip 53 provides a fluid-impermeable barrier between the reservoir and externally worn clothing, the antifungal agent is preferentially absorbed by the nail.

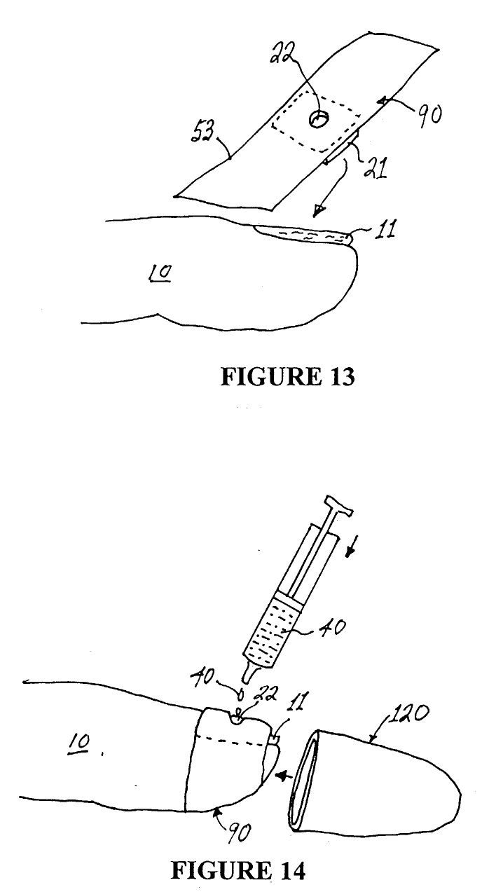

In summary, FIGS. 9-11 show the most preferred embodiment of a drug-dispensing dressing operable for administering a fluid containing an antifungal agent to a fungus infected nail on a toe or a finger. The dressing 90 comprises: (a) a reservoir 21 comprising a fluid absorbant pad having an upper surface, a nail-facing surface 91 in opposition to the upper surface and a first hole 22b therein; and (b) an elongate strip 53 of a fluid impermeable material having a second hole 22a in a central portion 92 thereof. The elongate strip 53 has a skin-facing surface 52 coated with a layer of adhesive. The skin-facing surface 52 of the central portion 92 is affixed to the upper surface of the fluid-absorbant pad 21 with the second hole 22a overlying and substantially coextensive with the first hole 22b. The two holes 22a and 22b are superimposed to form a single hole 22 through the dressing as shown in FIG. 13. The hole 22 is preferably about 2 millimeters deep with a diameter of about 4 millimeters to contain a drop of antifungal agent prior to absorption of the fluid into the reservoir material.

An elastic barrier suitable for use with any of the embodiments of the drug-dispensing dressings 20, 50 or 90 disclosed herein is illustrated in perspective view at numeral 120 in FIG. 12. The elastic barrier 120, in form, generally resembles a condom or a finger cot. The elastic barrier comprises a tubular elastomeric body 123, preferably silicone or latex, having an open end 121 and a closed end 122. The elastic barrier 120 is donned to overlie and envelop a drug-dispensing dressing applied to a digit such as a toe and provides an additional fluid-impermeable barrier to retain antifungal agent within the reservoir. When a dressing is applied to a toe, friction between the dressing and sock during walking has the effect of pulling the dressing off of the toe in a distal direction. The wall of the body 123, while elastic and flexible, is sufficiently thick (˜0.015 inch) so as to prevent the elastic barrier from moving in a distal direction or rolling upon itself (everting) once the foot is placed within a shoe.

FIG. 13 is a perspective view of a drug-dispensing dressing 90 in accordance with FIGS. 9-11 prior to being applied to a toe 10 having a nail 11 thereon that is infected with a fungus. FIG. 14 is a perspective view of a drug-dispensing dressing 90 in accordance with FIGS. 9-11 applied to a toe 10 with the fill hole 22 overlying the fungus infected nail 11. FIG. 14 shows an antifungal agent 40 being introduced into the reservoir 21 (not visible in FIG. 14) through the access hole 22 in the dressing 90, and a fluid impermeable barrier 120 readied for positioning on the toe to overlie the dressing 90 after the antifungal agent 40 is added to the reservoir.

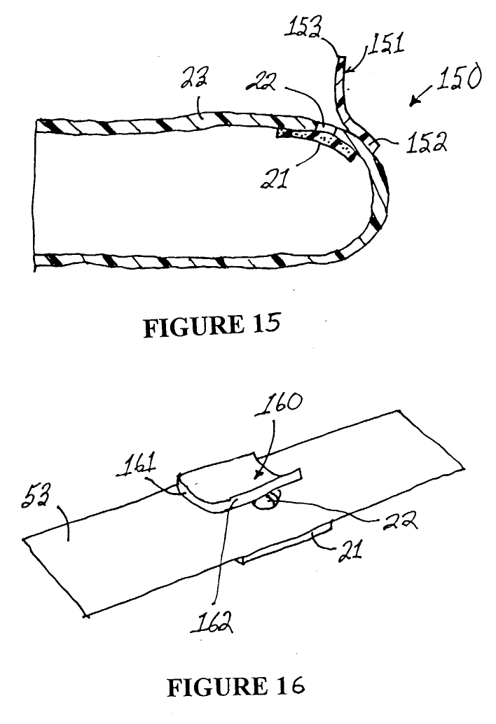

FIG. 15 is a longitudinal cross-sectional view of a drug-dispensing dressing 150 wherein the dressing 20 in accordance with FIGS. 2 and 3 is modified to further comprise an elastically deformable flap 151 having a fixed end 152 affixed to the outer surface of the dressing adjacent to the hole 22, and a free end 153. The flap is opened with the fingers to expose the hole 22 for filling the reservoir 21 with an antifungal agent.

FIG. 16 is a top perspective view of a drug-dispensing dressing in accordance with FIGS. 5-7 or 9-11 further comprising an elastically deformable flap 160 affixed to the strip 53 comprising the dressing at a fixed end 161 thereof. The flap 160 has a free end 162 and a flat body portion between the free end and the fixed end disposed to cover the hole 22. The flap is opened by raising the free end of the flap to expose the hole for filling the reservoir with an antifungal agent. After filling the reservoir, the flap returns to its normally closed position to overlie the hole.

Fluid Antifungal Compositions

As mentioned earlier, many antifungal compositions are known and more are presently being developed. Exemplary prior art antifungal agents include undecylenic acid, clotrimazole, sulconazole nitrate, terbinafine hydrochloride, ciclopirox olamine, miconazole, mycostatin, naftifine hydrochloride, griseofulvin, econazole nitrate and oxiconazole nitrate. Prior art compositions containing these antifungal agents and formulated for topical application are not effective for treating onychomycosis because they do not penetrate to the lower strata of the nail body where fungal mycelia and spores are present. Accordingly, much of the current research is directed toward identifying agents that increase the permeability of the nail body to the prior art antifungal agents.

A preferred composition for use with the drug-dispensing dressings described hereinabove for the treatment of onychomycosis is a clear, substantially transparent solution comprising a volume of 25% w/v undecylenic acid in isopropyl palmitate (Kramer Laboratories, Inc., Miami, Fla.) in an equal volume of a 1:1 (v/v) mixture of emu oil (Item No. UE1004, Uniquely Emu Products, Inc. Ottawa, Ill.) and tea tree oil (Item No. 400426, Health Enterprises, N. Attleboro, Mass.).

### EXAMPLE 1

A patient having onychomycosis of both the left and right great toe presented himself for treatment of tinea pedis. A course of treatment with an over-the-counter antifungal agent, self-administered, cured the tinea pedis within a month but the onychomycosis persisted for an additional 5 years. During this period, the patient periodically self-administered an over-the-counter solution containing undecylenic acid to the infected nails with a brush but the onychomycosis persisted. The patient was counseled regarding optional treatment but declined to risk the side-effects of a systemic antifungal agent.

A pair of drug-dispensing dressings were prepared by cutting two circular pieces of moleskin to a diameter of about 1.5 cm and adhering the adhesive-coated surface of the moleskin to the absorbant pad of a 1″×3″ NEXCARE™ bandage (3M Corporation), the moleskin providing an absorbant reservoir. A hole having a diameter of about 0.4 cm was punched through both the bandaid strip and the reservoir with a hole punch.

The patient's toes were wiped with alcohol and the nails clipped as short as practical. Burs were removed from the free end of the nails with an emery board and the reservoir was positioned centrally on the nail and the free ends of the adhesive strip adjacent to the central portion were wrapped around and adhered to the toe. About five drops of the preferred fluid antifungal composition containing about 12.5% undecylenic acid, made as described above, were placed in the hole directly onto the exposed uppermost surface of the nail and the solution was quickly absorbed and taken up by the reservoir. The dressings were covered with extra large finger cots and the patient provided with a drop dispenser comprising the preferred antifungal agent in a 3 ml syringe. The patient rolled down the finger cot to expose the hole and added drops of solution to the reservoir every 4 hours in an amount sufficient to restore reservoir saturation. The finger cot, which provided an additional barrier between the dressing and the patient's socks, were rolled then rolled up to cover the dressing. The dressing was removed after 3 days and no further treatment was provided. The finger cots, which were removed during hours of sleep, needed to be replaced each day due to deterioration from contact with the antifungal solution, but the original dressings remained in place throughout the course of treatment. Accordingly, a more resistant and durable removable barrier layer comprising a silicone finger cot is a preferred removable barrier.

A kit comprising a drug-dispensing dressing in accordance with any of the embodiments of the present invention, together with a fluid composition comprising an antifungal agent can be employed to treat onychomycosis. A preferred embodiment of the drug-dispensing dressing for inclusion within a kit is the tubular or “finger cot-like” embodiment 20 illustrated in FIGS. 2-4, either without a flap or with a flap as shown in FIG. 15. The embodiment 20 is preferable made from silicone rubber and may be molded or formed by repeatedly dipping a toe or finger-shaped mandrel into a dispersion of silicone elastomer and curing the coating of elastomer between dips to build up a wall thickness preferably 1 millimeter or greater. The elastic wall of the dressing can be stretched to place the dressing on the digit. The elastomeric wall enables the dressing to snugly conform to the contour of a toe. Fluid antifungal agents for use in the kit can be stored for dispensation either within a dropper bottle or a syringe. Suitable fluid antifungal agents include lavender oil, Miconazole, Clotrimazole, Tioconazole, Nystatin, Terconazole, Butoconazole Nitrate, Unecylenic Acid, Clioquinol, Ciclopirox Olamine, Econazole Nitrate, Triacetin, Tolnaftate, Flucytosine, Tea Tree Oil, Emu Oil, Ketoconazole or a composition comprising two or more of these antifungal agents.

While particular embodiments of the present invention have been illustrated and described, it would be obvious to those skilled in the art that various other changes and modifications can be made without departing from the spirit and scope of the invention. For example, the hole 22 in the strip 53 or elastic body 23 is preferably at least 3 millimeters in diameter in order to receive and contain a drop of fluid placed therein until it is absorbed by the reservoir. If, instead of a drop dispenser, a syringe is employed to inject a fluid composition into the reservoir, the hole is preferably smaller and dimensioned to snugly receive the male hub on the end of the syringe. It is therefore intended to cover in the appended claims all such changes and modifications that are within the scope of this invention.

What I claim is:

1. A drug-dispensing dressing for administering a fluid containing an antifungal agent to a fungus infected nail on a toe or a finger, the dressing comprising: (a) a reservoir comprising a fluid absorbant pad dimensioned to cover the infected nail and having an upper surface and a nail-facing surface in opposition to said upper surface; and (b) an elongate strip of a fluid impermeable material having a hole in a central portion thereof and having a diameter of at least 3 millimeters, said elongate strip having a skin-facing surface coated with a layer of adhesive, and an upper surface in opposition to said skin-facing surface, said skin-facing surface of said central portion being affixed to said upper surface of said fluid-absorbant pad with said hole overlying said pad.

2. The drug-dispensing dressing of claim 1 wherein said elongate strip further comprises a flap made of a fluid-impermeable material, said flap having a fixed end affixed to said central portion of said upper surface thereof, and a free end in opposition to said fixed end, and a flap body therebetween, wherein said flap body removably covers said hole.

3. A drug-dispensing dressing for administering a fluid containing an antifungal agent to a fungus infected nail on a toe or a finger, the dressing comprising: (a) a reservoir comprising a fluid absorbant pad having an upper surface, a nail-facing surface in opposition to said upper surface and a first hole therein; and (b) an elongate strip of a fluid impermeable material having a second hole in a central portion thereof, said elongate strip having a skin-facing surface coated with a layer of adhesive, and an upper surface in opposition to said skin-facing surface, said skin-facing surface of said central portion being affixed to said upper surface of said fluid-absorbant pad with said second hole overlying and substantially coextensive with said first hole.

4. The drug-dispensing dressing of claim 3 wherein said elongate strip comprises a flap having a fixed end affixed to said central portion of said upper surface thereof, and a free end in opposition to said fixed end, and a flap body therebetween, wherein said flap body removably covers said second hole.

5. A drug-dispensing dressing for administering a fluid containing an antifungal agent to a fungus infected nail on a toe or a finger, the dressing comprising: (a) a tubular elastic member having an outer surface, an open proximal end and a closed distal end and a substantially cylindrical interior cavity therebetween, said interior cavity having a cavity wall, said cavity wall having a hole therein adjacent to said distal end, said hole providing fluid communication between said outer surface and said cylindrical interior cavity; and (b) a reservoir comprising an absorbant pad operable for absorbing said fluid containing said antifungal agent affixed to said cavity wall and underlying said hole.

6. The drug-dispensing dressing of claim 5 wherein said absorbant pad has a second hole therein that underlies said hole in said cavity wall.

7. The drug-dispensing dressing of claim 6 wherein said elongate strip comprises a flap having a fixed end affixed to said central portion of said upper surface thereof, and a free end in opposition to said fixed end, and a flap body therebetween, wherein said flap body removably covers said second hole.

8 A method for treating fungal infections of the nail comprising the steps of: (a) presenting a drug-dispensing dressing in accordance with claim 5 and placing the dressing over the affected digit such that the reservoir overlies the infected nail; then (b) applying a solution to the reservoir consisting essentially of lavender oil and an anti-fungal agent selected from the group consisting of Miconazole, Clotrimazole, Tioconazole, Nystatin, Terconazole, Butoconazole Nitrate, Unecylenic Acid, Clioquinol, Ciclopirox Olamine, Econazole Nitrate, Triacetin, Tolnaftate, Flucytosine, Tea Tree Oil, Emu Oil and Ketoconazole.

9 A method for treating fungal infections of the nail comprising the steps of: (a) presenting a drug-dispensing dressing in accordance with claim 6 and placing the dressing over the affected digit such that the reservoir overlies the infected nail; then (b) applying a solution to the reservoir consisting essentially of lavender oil and an anti-fungal agent selected from the group consisting of Miconazole, Clotrimazole, Tioconazole, Nystatin, Terconazole, Butoconazole Nitrate, Unecylenic Acid, Clioquinol, Ciclopirox Olamine, Econazole Nitrate, Triacetin, Tolnaftate, Flucytosine, Tea Tree Oil, Emu Oil and Ketoconazole.

10 A kit for treating onychomycosis of a nail on a digit comprising: (a) a device operable for dispensing a therapeutically effective dosage of a fluid antifungal composition to the infected nail on the digit comprising an elastically extensible fluid-impermeable tubular member having an open proximal end and a sealed distal end, and a body portion therebetween with a hole therein, said tubular member having a substantially cylindrical interior wall with a reservoir comprising a fluid absorbing pad affixed thereto, the reservoir disposed to underlie said hole in said body portion; and (b) a fluid antifungal composition contained within a dispenser.

11 The kit of claim 10 wherein said dispenser is a drop dispenser.

12 The kit of claim 10 wherein said dispenser is a syringe.

13 The kit of claim 12 wherein said syringe has a male hub extending from a distal end thereof and wherein said hole in said body portion of said device is dimensioned to snugly receive a distal portion of said hub.
